(19) United States
(12) Patent Application Publication
(10) Pub. No.: US 2021/0244576 A1
(43) Pub. Date: Aug. 12, 2021

(54) FOLDED ABSORBENT ARTICLE

(71) Applicant: Essity Hygiene and Health Aktiebolag, Goteborg (SE)

(72) Inventors: Robert Albino, Goteborg (SE); Erik Jamsa, Goteborg (SE)

(21) Appl. No.: 17/252,794

(22) PCT Filed: Jun. 26, 2018
(86) PCT No.: PCT/SE2018/050683

§ 371 Date: Dec. 16, 2020

(51) Int. Cl. A61F 13/49; A61F 13/56; A61F 13/531; A61F 13/551; A61F 13/15

(52) U.S. Cl. CPC A61F 13/49001; A61F 13/5633; A61F 13/531; A61F 2013/530489; A61F 13/15747; A61F 13/15756; A61F 13/15203; A61F 13/55115

(57) ABSTRACT

A folded absorbent article includes an absorbent core sandwiched between a liquid-permeable topsheet and a liquid-impermeable backsheet. The absorbent article defines, in an unfolded and planar state, a longitudinal center line and a transverse center line. Each transverse side of the absorbent article is folded along a respective longitudinal fold line, such that each of first and second transversely opposed side edges are folded onto a topsheet surface of the absorbent article thereby forming a firstly folded absorbent article. The firstly folded absorbent article is thereafter folded along a first transverse fold line located in the region of the transverse center line thereby forming a secondly folded absorbent article. Finally, the secondly folded absorbent article is folded along a second transverse fold line located approximately in the middle between the transverse center line and a waist edge thereby forming a thirdly folded absorbent article.

### CROSS-REFERENCE TO RELATED APPLICATION

This application is a national phase entry of, and claims priority to, International Application No. PCT/SE2018/050683, filed Jun. 26, 2018. The above-mentioned patent application is incorporated herein by reference in its entirety.

### TECHNICAL FIELD

This application is related to a folded absorbent article comprising an absorbent core that has a first and a second primarily longitudinally extending channel located at least in the crotch portion of the absorbent article, as well as a corresponding method for manufacturing such a folded absorbent article.

### BACKGROUND

In the field of absorbent articles, in particular open diapers and open baby diapers, there is a demand for providing a package of stacked absorbent articles that has a relatively symmetrical and stable outer shape for enabling improved handling and storing of the package and for providing an aesthetically attractive package.

Moreover, there is also a demand for providing a package of stacked absorbent articles in which a stack quality of the individual absorbent articles within the package is high, such that individual absorbent articles that are taken from the package has an appealingly appearance and are free from unintended folds that may have detrimental effect on absorption performance and leakage security.

Despite the activities in the field, there is still a demand for providing an improved package of stacked absorbent articles, and improved absorbent articles for enabling a better package, in terms of symmetrical, stable and aesthetically attractive packages, stack quality and quality of individual absorbent articles that are taken from the package.

There is continuous development towards thinner and more pliable absorbent articles because these characteristics generally results in improved wearing comfort, user fitting and discretion. However, thin and pliable absorbent articles are generally more problematic in terms of stacking and packaging of the stacked absorbent articles. Relatively thin and pliable absorbent articles have less internal structural rigidity and are therefore frequently unintentionally slightly misplaced or folded during the stacking and packaging process.

For example, one type of stacking and packaging process for absorbent articles involves placing a set of finished and bi-folded absorbent articles next to one another in individual compartments of a packaging tool, wherein the individual compartments are designed and oriented such that the set of absorbent articles resembles a stack. When each compartment is filled with an individual absorbent articles the packaging tool compresses the stack in the direction of the stack and pushes the stack of absorbent articles into a plastic bag that is subsequently closed to form a finished package.

However, the process step of inserting the stack of thin and pliable absorbent articles into the bag is problematic due to the relatively thin and pliable absorbent articles. In particular the non-folded edge of a bi-folded absorbent article generally causes packaging problems due to the low internal stability. Bi-folded absorbent articles are absorbent articles that have been folded once along a central fold line that extends perpendicular to the length direction of the flattened absorbent article, such that a topsheet of a rear portion after folding faced the topsheet of the front portion of the absorbent article. The non-folded edge of a bi-folded absorbent article is particularly structurally instable due to the lack of a folded edge at the non-folded end and due to the lack of a core at the front and rear waist edge, which form the non-folded edge of a bi-folded absorbent.

As a result, some absorbent articles may be slightly displaced with respect to the other absorbent articles in the stack because the thin and pliable absorbent articles tend to become deformed, folded, wrinkled, etc. instead of being properly pushed into the package during the step of pushing the stack of absorbent articles into a plastic bag.

This uncontrolled behavior of the individual absorbent articles during packaging process may result in reduced stack quality in terms of poor alignment of individual absorbent articles in the stack, less appealingly appearance due to unintended wrinkles or folds, increased interference with adjacent absorbent articles in the stack during removal of the absorbent article from the stack, detrimental effect of absorption capacity due to unintended folds of the absorbent core.

Moreover, stacking of thin and pliable absorbent articles tend to render the stack less stable, such that the overall shape of the finished and packaged package may deviate from a symmetrical shape. For example, the non-folded edge of a bi-folded thin and pliable absorbent article is particularly structurally instable and may result in a curved stacking axis due to the smaller thickness of the absorbent articles in the non-folded edge compared with the thickness of the folded edge.

One feature that generally results in significantly reduced structural rigidity and increased pliability of an absorbent article is channels provided in the absorption core of the absorbent article, in particular longitudinally extending channels.

Longitudinally extending channels may improve insulting fluid distribution along the length of the channel, such that a wider area of the core is used for fluid absorption and a better utilization of the absorbent capacity of the core is accomplished. The channels may also provide increased fluid acquisition speed.

Therefore, it would be desirable to provide an absorbent article, which despite being relatively pliable and structurally weakened by longitudinally channels in the absorbent core, enables improved packaging of stacked absorbent articles into a consumer package in terms of symmetrical, stable and aesthetically attractive packages, stack quality and quality of individual absorbent articles that are taken from the package.

### SUMMARY

In order to achieve these technical objectives, this application provides, in several embodiments, a folded absorbent article, a package comprising such a folded absorbent article and a method for manufacturing such a folded absorbent article.

According to a first embodiment, there is provided a folded absorbent article comprising an absorbent core sandwiched between a liquid-permeable topsheet and a liquid-impermeable backsheet. The absorbent article comprises, in an unfolded and planar state, a longitudinal center line, a transverse center line, a longitudinal length extending from a front waist edge to a back waist edge, and a transverse length extending from a first side edge to a second side edge. The absorbent article further comprises a front portion, a back portion and a crotch portion, and fastening tabs located on each transverse side of the back portion for being releasably fastened to the front portion when the absorbent article is in a fastened position. The absorbent core comprises a first and a second primarily longitudinally extending channel located at least in the crotch portion. Each transverse side of the absorbent article is folded along respective longitudinal fold line, such that each of first and second transversely opposed side edges are folded onto a topsheet surface of the absorbent article thereby forming a firstly folded absorbent article. The firstly folded absorbent article is folded along a first transverse fold line located in the region of the transverse center line thereby forming a secondly folded absorbent article. Finally, the secondly folded absorbent article is folded along a second transverse fold line located approximately in the middle between the transverse center line and a waist edge thereby forming a thirdly folded absorbent article.

According to a second embodiment, there is provided a method for manufacturing a folded absorbent article, the absorbent article comprising, in an unfolded and planar state, a longitudinal center line, a transverse center line, a longitudinal length extending from a front waist edge to a back waist edge, and a transverse length extending from a first side edge to a second side edge, said absorbent article further comprising a front portion, a back portion and a crotch portion, and fastening tabs located on each transverse side of the back portion for being releasably fastened to the front portion when the absorbent article is in a fastened position, the method comprising: forming an absorbent core comprising a first and a second primarily longitudinally extending channels located at least in the crotch portion; sandwiching the absorbent core between a liquid-permeable topsheet and a liquid-impermeable backsheet, folding each transverse side of the absorbent article along respective longitudinal fold line, such that each of first and second transversely opposed side edges are folded onto a topsheet surface of the absorbent article thereby forming a firstly folded absorbent article; folding the firstly folded absorbent article along a first transverse fold line located in the region of the transverse center line thereby forming a secondly folded absorbent article; and66 folding the secondly folded absorbent article along a second transverse fold line located approximately in the middle between the transverse center line and a waist edge thereby forming a thirdly folded absorbent article.

One solution to the above-mentioned problems is thus to apply quattro folding of the absorbent article because thereby the internal structural rigidity of the finished and folded absorbent article is significantly increased. In particular, the previous weak non-folded edge of a bi-folded absorbent article is substantially strengthened in terms of structural rigidity when applying quattro folding technique.

The term “quattro folding” herein refers to folding of an absorbent article such that a central core of the absorbent article has been folded twice along transverse fold lines, i.e. such that the absorbent article has a total of four overlapping panels of the absorbent article. An unfolded article has a single panel.

When each of first and second transversely opposed side edges have been folded onto a topsheet surface of the absorbent article to form the firstly folded absorbent article, and when the firstly folded absorbent article has been folded along the first transverse fold line located in the region of the transverse center line to form the secondly folded absorbent article, the secondly folded absorbent article may be deemed to have two overlapping sections of the absorbent article, i.e. defining a bi-folded absorbent article.

Furthermore, when the secondly folded absorbent article is folded along the second transverse fold line located approximately in the middle between the transverse center line and the waist edge to form the thirdly folded absorbent article, the thirdly folded absorbent article may be deemed to have four overlapping sections of the absorbent article, i.e. defining a quattro folded absorbent article.

The quattro folded absorbent article forms a compact unit with high structural rigidity in all directions, since all edges of the quattro folded article in fact includes a folded edge. Both the unfolded front and back waist edge are located together with the folded edge of the first transverse fold line, the folded edge of the second transverse fold line is located at the oppositely located edge of the quattro folded article, and each of the side edges quattro folded article include a longitudinal fold line.

Moreover, the four overlapping sections of the absorbent article of approximately equal size efficiently stabilizes weak areas within the absorbent article, such as area of the first and second channels.

As a result of the increased structural rigidity of the quattro folded article the handling and position control of the folded absorbent article during the packaging process can be improved, and the risk for unsatisfactory alignment of individual absorbent articles in the stack, less appealingly appearance, interference with adjacent absorbent articles in the stack during removal, detrimental effect of absorption capacity, and unsymmetrical shape of the overall shape of the finished and packaged package, is reduced.

Moreover, the structure rigidity of the folded absorbent article is increased because the core is then present at all parts of the folded article to a larger degree, thereby also providing a thicker folded absorbent article.

In addition, quattro folding results in smaller and thicker folded absorbent articles, compared with bi-folded absorbent articles or non-folded absorbent articles. As a result, there is increased flexibility in terms of arranging the folded absorbent articles within the package. This flexibility may for example be exploited for reducing the amount of package material required for the package with maintained number of folded absorbent articles within the package, or for providing a package with a larger front face surface area for increased brand exposure in the shelf of a warehouse. In addition, this flexibility may further be exploited for providing increased self-standing stability, or for increasing the packaging utilization on a standard size transportation pallet.

Moreover, in case of single-wrap of each individual folded absorbent article, i.e. where each folded absorbent article is wrapped in an individual wrapping, the amount of material required for each individual wrapping is reduced because the shape of a quattro folded absorbent article is closer to the optimal form of a sphere compared with a bi-folded absorbent article that typically has a more flat, spread out and sheet-like outer geometry.

The smaller and thicker quattro folded absorbent articles also simplifies bring along of an individual absorbent article because it also fits into a relatively small purse or bag.

The second transverse fold line does not intersect a front portion of any of the first and second channels. As mentioned above, the first and second channels serve at least partly to act as fluid conveying channels for distributing insulting fluid over a wider area of the core for improved utilization of the total absorbent capacity of the core, as well as for increasing the fluid acquisition speed. However, if a fold of the absorbent article along the second transverse fold line intersect with any of the first or second channels the fold may cause deformation, damage or hardening of the core at the intersection, such that the desired fluid conveying functionality is damaged and degraded. Consequently, by locating the second transverse fold line such that it does not intersect a front portion of any of the first and second channels, the risk that a fold of the absorbent article along the second transverse fold line negatively interfere with the fluid conveying performance of the first and second channels is reduced.

The second transverse fold line does not intersect a back portion of any of the first and second channels. Similar to above, by locating the second transverse fold line such that it does not intersect a back portion of any of the first and second channels, the risk that a fold of the absorbent article along the second transverse fold line negatively interfere with the fluid conveying performance of the first and second channels is reduced.

An inner surface of the back portion may face an inner surface of the front portion of the secondly folded absorbent article, and an outer surface of the back portion may face an outer surface of the crotch portion of the thirdly folded absorbent article. This folding structure of an initially substantially planar absorbent article represents one variant out of a total of four available variants, each representing a unique final folding structure, of a quattro folded absorbent article that is first folded along a first central transverse fold line and subsequently folded along a second transverse fold line located approximately in the middle between the transverse center line and a waist edge, ignoring the longitudinal fold line along each transverse side of the absorbent article.

This particular variant, which has an inner surface of the back portion facing an inner surface of the front portion in the secondly folded absorbent article, ensures that the backsheet faces outwards and the topsheet, which will face the skin of the user, is better protected from dirt etc., thereby providing a more hygienic absorbent article.

Moreover, by subsequently selecting to have the outer surface of the back portion facing the outer surface of the crotch portion of the thirdly folded absorbent article, it is ensured that an outer front region of absorbent article remains visible on the quattro folded absorbent article. This is beneficial because the outer front region is typically used for informative printing, such as for example size of the absorbent article, and brand of the producer of the absorbent article. Hence, a consumer may still in a folded state of the thirdly folded absorbent article, i.e. quattro folded state, acquire relevant information about for example size, type and/or brand of the absorbent article, thereby reducing the need for unfolding the quattro folded article for acquiring this information.

A further advantage of having the inner surface of the back portion facing the inner surface of the front portion of the secondly folded absorbent article, and having the outer surface of the back portion facing the outer surface of the crotch portion of the thirdly folded absorbent article, is improved pre-forming of the absorbent article for better fit on the body of the user. A pre-formed absorbent article for better fit on the body of the user has typically slightly curved natural shape along the longitudinal center line with the topsheet facing towards a radially inner side of the curved shape. Such a curved natural shape is partly provided by folding the absorbent article along the first and second transverse fold lines as defined above.

Folding along the first transverse fold line to have the inner surface of the back portion facing the inner surface of the front portion of the secondly folded absorbent article creates a clear and desirable pre-forming of the article in crotch portion towards a having a curved shape with the topsheet located on the radially inner side.

Moreover, folding along the second transverse fold line to have the outer surface of the back portion facing the outer surface of the crotch portion of the thirdly folded absorbent article creates a pre-forming of the article towards a having a curved shape with the topsheet located on the radially inner side in the front portion of the article but the topsheet located on the radially outer side in the back portion of the article. However, since the core of the absorbent article typically extends closer to the front waist edge than the rear waist edge, and since the absorbent core may have a reduced thickness in back portion, the desired pre-forming effect, i.e. having the topsheet located on the radially inner side, caused by folding along the second transverse fold line is relatively large in the front portion of the article, and the undesirable pre-forming effect, i.e. having the topsheet located on the radially outer side, at the second transverse fold line is relatively small in the back portion of the article. To conclude, the specific quattro folding results in a desirable pre-forming effect on the article in the front and crotch portion, while the undesirable pre-forming effect in the back portion generally is relatively weak due to reduced thickness or lack of absorbent core in the back portion.

Still a further advantage of the above-defined specific quattro folding is that folding of the article along second transverse fold line results in a smaller radius fold at the back portion and a larger radius fold at the front portion since the front portion of the article surrounds the back portion, as seen from a lateral side of article. This is advantageous because the back portion of the article typically has a smaller thickness than the front portion, partly due to the aforementioned forwards located core and reduced thickness of the absorbent core in the back portion, thereby enabling the back portion to more easily and with less risk for damages to the absorbent core have a smaller radius fold than the front portion, and thus providing an overall thinner folded absorbent article.

At least one of the first and second channels may have a length which is between 5-50%, specifically 10-50%, and more specifically 28-38%, of a total length of the absorbent article. These dimensions have shown provide a satisfactory fluid distribution effect in the core.

At least one of the first and second channels may have a length which is between 10-60%, specifically between 20-60%, and more specifically between 30-50%, of a length of the absorbent core. These dimensions have shown provide a satisfactory fluid distribution effect in the core.

A distance between the front waist edge of the article and a front edge of at least one of the first and second channels may be between 15-40%, and specifically between 22-25%, of a total length of the article. These dimensions have shown provide a satisfactory fluid distribution effect in the core.

The first and second channels may be generally straight. This shape of the channels provide improved fluid distribution effect in the core.

The first and second channels may be generally parallel to said longitudinal center line. This orientation of the channels provide improved fluid distribution effect over the whole length of the core.

The first and second channels may constitute sections of the absorbent core which are generally free from absorbent material. Thereby insult fluid may easily flow along the channels.

The first and second channels may constitute sections of the absorbent core which have a thinner layer of absorbent material than adjacent areas of the absorbent core. Thereby, the sections of the core which form the channels are more quickly soaked and may more quickly convey fluid to adjacent areas of the core.

Each of the first and second channels may have a width of at least 3 mm, specifically at least 4 mm, and more specifically at least 5 mm. These dimensions have shown provides satisfactory fluid distribution effect in the core.

The absorbent core may comprise additional primarily longitudinally extending channels located at least in the crotch portion. In other words, the absorbent core may comprise also a third, or even more, primarily longitudinally extending channel located in the crotch portion. The absorbent core may further comprise one or more additional channels extending in other directions and/or being located in the back and/or front portion of the core. Additional channels may in certain situations and implementations have beneficial effect on fluid absorption and leak security.

The absorbent core may, in a region of the absorbent core without a channel, comprise about 60-100 wt % SAP. If the absorbent core in a region of the absorbent core without a channel comprises less than 100 wt % SAP the remaining material may for example be primarily pulp material, or the remaining material may for example be only pulp material. A relatively high SAP/pulp ratio indicates a thin absorbent article and thus a relatively pliant absorbent article. Hence, quattro folding is particularly advantageous when the absorbent core in a region without a channel or hole comprises about 60-100 wt % SAP.

A thickness of the absorbent article in an unfolded state and in a region of the absorbent core may be less than 7 mm, specifically less than 6 mm, and more specifically less than 5 mm. A thinner absorbent product is typically more pliant than a thick product. Hence, the quattro folding is particularly advantageous for absorbent articles having a thickness in the area of the core of less than 7 mm. A method for measuring the thickness of the absorbent article is described in detail further down in the disclosure.

A density of the absorbent article 1 in the region of the absorbent core is higher than 0.16 g/cm³, specifically higher than 0.18 g/cm³, and more specifically higher than 0.20 g/cm³. A high density is an indicator of high SAP/pulp ratio, and thus relatively low level of a voluminous fluff pulp. In other words, a high density is an indicator of a relatively pliant absorbent article, which thus benefits from quattro folding. A method for measuring the density of the absorbent article is described in detail further down in the disclosure

The absorbent core may be constituted of one single core layer. This enables simplified manufacturing of the absorbent article.

The absorbent core may have a generally rectangular shaped, as seen from a top of the absorbent article in a flat state. This enables less scrap material when cutting the absorbent core from a continuous strip of absorbent material.

The absorbent article may be an open baby diaper absorbent article. These type of articles are generally relatively small and thereby particularly difficult to pack high stack quality and without unintended folds and wrinkles.

In another embodiment, a package is provided comprising folded absorbent article as described above, wherein the package comprises at least two parallel stacks, specifically at least three parallel stacks, and more specifically at least four parallel stacks of folded absorbent articles located side-by-side. As described above, increased number of parallel stacks enables outer package dimensions that are less narrow and more resembling a cubical shape, thereby enabling increased front face surface for displaying size and brand, as well as providing a more self-stable article, especially with having relatively low number of articles in each package.

Further features of, and advantages with, the present disclosure will become apparent when studying the following description. The skilled person realize that different features of the present disclosure may be combined to create embodiments other than those described in the following, without departing from the scope of the present disclosure.

### BRIEF DESCRIPTION OF THE DRAWINGS

The features and advantages of the invention will be appreciated upon reference to the following drawings. The accompanying drawings, which are incorporated in and constitute a part of this specification, illustrate one or more embodiments of the invention and, together with the general description given above and the detailed description given below, explain the one or more embodiments of the invention.

FIG. 1 is a top perspective view of a bi-folded absorbent article with core channels.

FIG. 2 is a top perspective view of the absorbent article of FIG. 1 when axial compression forces are exerted in the article.

FIG. 3 is a top view of an absorbent article in a flat state and with core channels.

FIG. 4 is a top view of the absorbent article of FIG. 3 after folding into a firstly folded absorbent article according to one embodiment.

FIG. 5 is a top view of the absorbent article of FIG. 3 after folding into a secondly folded absorbent article.

FIG. 6 is a top view of the absorbent article of FIG. 3 after folding into a thirdly folded absorbent article.

FIG. 7 is a top perspective view of an absorbent article with core channels in a flat state corresponding to FIG. 3.

FIG. 8 is atop perspective view of the absorbent article of FIG. 7 after folding into a firstly folded absorbent article.

FIG. 9 is atop perspective view of the absorbent article of FIG. 7 after folding into a secondly folded absorbent article.

FIG. 10 is a top perspective view of the absorbent article of FIG. 7 after folding into a thirdly folded absorbent article.

FIG. 11 is a side view of an absorbent article in quattro folded state.

FIG. 12 is a perspective view of an example of a pre-formed natural state of the article of FIG. 11 after unfolding.

FIG. 13 is a perspective view of an example of open diaper in an open state that may be folded according to one embodiment.

FIG. 14 is a top perspective view of the diaper of FIG. 13 in a closed state.

FIG. 15 is a top view of an example embodiment of a package of folded absorbent articles according to another embodiment.

FIG. 16 is a top view of a further example embodiment of a package of folded absorbent articles according to a further embodiment.

FIG. 17 is a side cross-section of the package of FIG. 16, taken along line A-A in that FIG. 16.

FIG. 18 is a side cross-sectional view of the package of FIG. 17 in an open state.

FIG. 19 is a top view of the absorbent article in a flat state and with a plurality of samples illustrated.

### DETAILED DESCRIPTION

The present folded absorbent article and associated package will now be described more fully hereinafter with reference to the accompanying drawings, in which exemplary embodiments of the disclosure are shown. The folded absorbent article and associated package may, however, be embodied in many different forms and should not be construed as limited to the embodiments set forth herein; rather, these embodiments are provided for thoroughness and completeness. Like reference characters refer to like elements throughout the description. The drawings are not necessarily to scale and certain features may be exaggerated in order to better illustrate and explain the exemplary embodiments of the present disclosure.

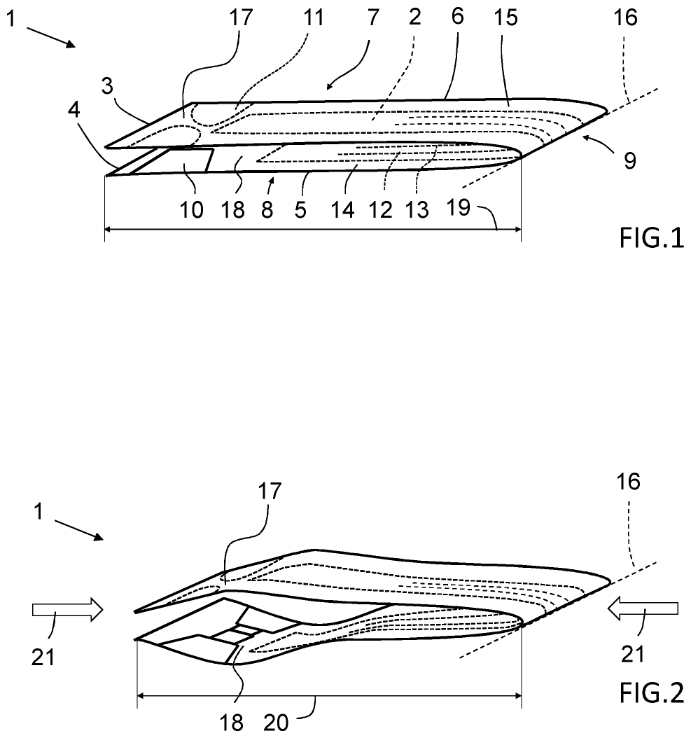

With initial reference to FIG. 1 there is shown a schematic 3D-view of an example embodiment of a bi-folded open absorbent article 1 comprising an absorbent core 2 with a first and a second channels 12, 13, a liquid-permeable topsheet 14 and a liquid-impermeable backsheet 15, a front waist edge 3 and a back waist edge 4. The absorbent article 1 further comprising a front portion 7, a back portion 8, a crotch portion 9, and fastening tabs 10 located on the back portion 8 and side panels 11 on the front portion 7.

The topsheet 14 is arranged at the surface of the article, i.e. at the side facing the wearer, whereas the backsheet 15 is arranged at the underside of the article 1. Furthermore, both the topsheet 14 and the backsheet 15 may extend laterally outside of the absorbent core 2 along the entire perimeter of the article 1.

The absorbent article has been folded along a centrally located first transverse fold line 16 to have two overlapping sections 17, 18 of the absorbent article 1 of approximately equal size, thereby forming the bi-folded absorbent article 1 having a first longitudinal length 19.

The first and second channels 12, 13 serve as insult fluid conveying channels for enabling more swiftly spreading of insult liquid over a larger area of the absorbent core 2. However, the first and second channels 12, 13 tend to reduce the structural strength of the absorbent article 1, and thereby making the absorbent article more floppy and pliable. This may be particularly problematic during manufacturing and packaging of individual absorbent articles 1 in stacks enclosed by a bag.

For example, upon placing a set of individual articles 1 in stack and subsequently compressing the stack and pushing the stack into an empty bag the risk for damages to the absorbent articles 1 in terms of unintended folds and wrinkles to the absorbent articles is increased when the absorbent articles have less internal structural strength.

Moreover, the manufacturing step of placing a set of individual articles 1 in stack in an aligned manner to provide a high stack quality may also be problematic because the relatively pliable absorbent articles 1 may more easily deform during handing by the mechanical manufacturing equipment that packages stacks of individual absorbent articles in bags.

For example, one type of stacking and packaging process for absorbent articles involves placing a set of finished and bi-folded absorbent articles next to one another in individual compartments of a packaging tool, wherein the individual compartments are designed and oriented such that the set of absorbent articles resembles a stack. When each compartment is filled with an individual absorbent article the packaging tool compresses the stack in the direction of the stack and pushes the stack of absorbent articles into a plastic bag that is subsequently closed to form a finished package.

However, some individual absorbent articles of the stack may be slightly deformed upon being placed in the individual compartment, such as for example schematically illustrated in FIG. 2, where the bi-folded absorbent article 1 has become slightly compressed in a longitudinal direction to have a second longitudinal length 20 that is smaller than the first longitudinal length 19. Such deformation may for example be caused by an axial force 21 that is exerted on the bi-folded absorbent article 1 in the longitudinal direction. This has a detrimental effect on stack quality because the alignment of individual absorbent articles 1 within the stack will then be degraded.

Consequently, some absorbent articles may be positioned slightly offset with respect to the other absorbent articles in the stack and this typically causes some absorbent articles to be unintentionally folded at an edge of the article. The uncontrolled behavior of the individual absorbent articles during packaging process caused by the increased pliability from the channels thus potentially results in reduced stack quality, less appealingly appearance due to unintended wrinkles or folds, and possibly even detrimental effect of absorption capacity due to unintended folds of the absorbent core.

The solution to this problem is to apply so called quattro folding on the absorbent article for improving the structural rigidity of the folded absorbent article and thereby improving handling and packaging quality of the absorbent article. The new folding according to the disclosure is particularly suitable for solving the problem of handling of absorbent articles with a first and second channel in the absorbent core during manufacturing, because the additional folding has a strengthening effect on the folded absorbent article 1.

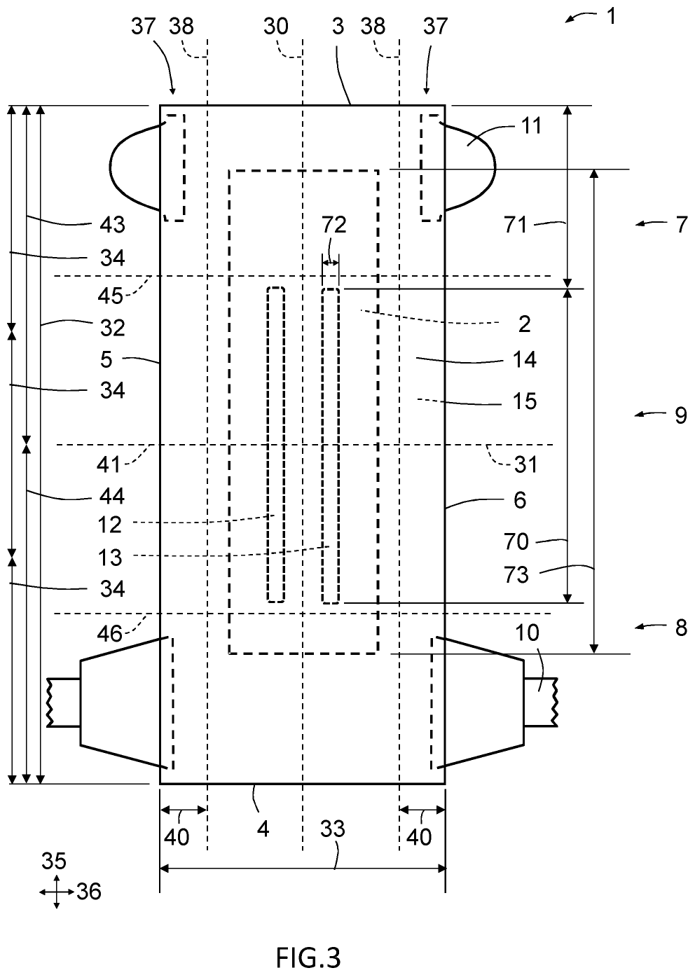

An example embodiment of the absorbent article 1 will now be described in detail with reference to FIGS. 3. The absorbent article 1 comprises an absorbent core 2 sandwiched between a liquid-permeable topsheet 14 and a liquid-impermeable backsheet 15. The absorbent article comprises, in the illustrated unfolded and planar state, a longitudinal center line 30, a transverse center line 31, a longitudinal length 32 extending from a front waist edge 3 to a back waist edge 4, and a transverse length 33 extending from a first side edge 5 to a second side edge 6 in a transverse direction 36.

The absorbent article further comprises a front portion 7, a back portion 8 and a crotch portion 9, each having the same length 34 in the longitudinal direction 35. The absorbent article 1 further comprises fastening tabs 10 located on each transverse side edge 5, 6 of the back portion 8 for being releasably fastened to the front portion 7 when the absorbent article is in a fastened position on a user, and side panels 11 on the front portion 7. The absorbent core 2 comprises a first and a second primarily longitudinally extending channel 12, 13 located at least in the crotch portion 9 of the absorbent article 1.

The absorbent core 2 shown in FIG. 1 has a rectangular design. However, the disclosure is not limited to this design but may be formed in generally any geometric form within the scope of the disclosure. Moreover, the absorbent core 2 may be constituted of one single core layer, or two or more stacked core layers.

The first and second channels 12, 13 are illustrated having the same length, width and form. The first and second channels 12, 13 are position in a central region of the absorbent article 1 and symmetrically on each side of the longitudinal center line 30.

Each of the first and second channel 12, 13 may have a length 70 which is between 5-50%, specifically 10-50%, and more specifically 28-38%, of the total length 32 of the absorbent article 1.

Moreover, each of the first and second channels 12, 13 have a length 70 which is between 10-60%, specifically between 20-60%, and more specifically between 30-50%, of a length 73 of the absorbent core 2.

In addition, a distance 71 between the front waist edge 3 of the absorbent article 1 and a front edge of the first and second channels 12, 13 is between 15-40%, and specifically between 22-25%, of a total length 32 of the absorbent article 1.

In certain example embodiments, each of the first and second channels 12, 13 has a width 72 of at least 3 mm, specifically at least 4 mm, and more specifically at least 5 mm, as seen in the transverse direction 36.

In certain example embodiments, the first and second channels 12, 13 have are generally straight shape. Moreover, the first and second channels 12, 13 may have oriented with their longitudinal axis generally parallel to the longitudinal center line 30 of the absorbent article 1.

The first and second channels 12, 13 may have various design and compositions. For example, the first and second channels 12, 13 may constitute sections of the absorbent core 2 which are generally free from absorbent material. This may for example be realized by cutting out channels in a finished absorbent core. Alternative, this may be obtained through manufacturing the absorbent core 2 involving a mat forming process during which absorbent material is omitted from the areas which correspond to the channels 12, 13. In this manner, no absorbent material will be present in the channels 12, 13.

Alternatively, the first and second channels 12, 13 may constitute sections of the absorbent core 2 which have a thinner layer of absorbent material than adjacent areas of the absorbent core 2. The thickness is measured in a direction 46 perpendicular to the longitudinal and transverse direction 35, 36.

The absorbent article 1 according to the example embodiment illustrated in FIG. 3 comprises two channels, namely the first and second channels 12, 13. However, the absorbent article may alternatively comprise further channels, such as for example a total of three, four, five or more channels. The further channels may extend along a primarily longitudinal direction 35, or alternatively in other directions.

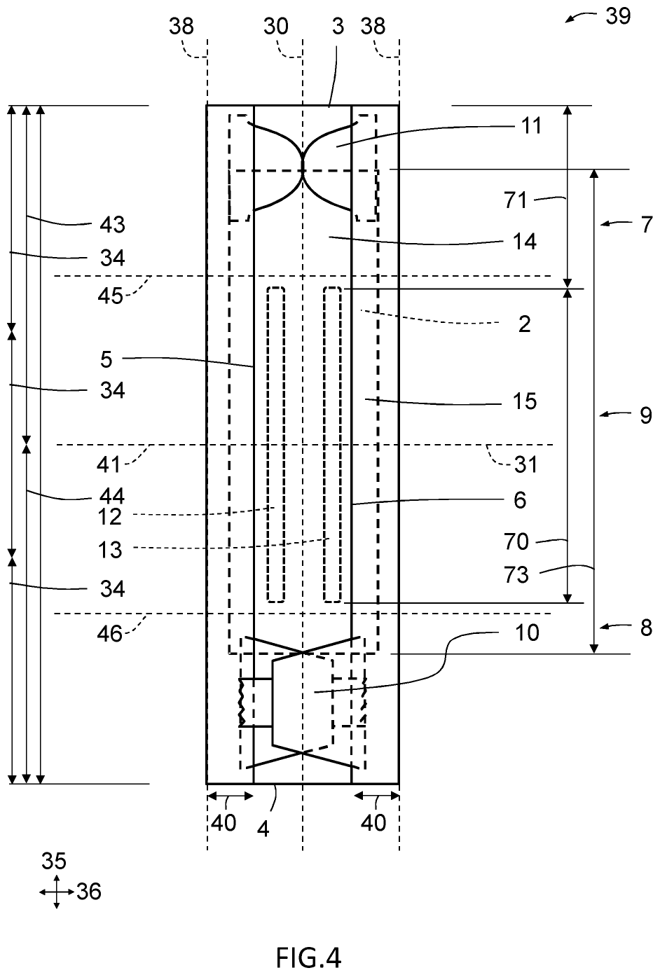

A folding of the absorbent article shown in FIG. 3 will now be described in detail with reference to FIGS. 4-6, which schematically shows different folding stages of the absorbent article 1.

In a first folding step each transverse side 37 of the absorbent article 1 is folded along respective longitudinal fold line 38, such that each of first and second transversely opposed side edges 5, 6 are folded onto a topsheet surface of the absorbent article 1 thereby forming a firstly folded absorbent article 39. The firstly folded absorbent article 39 is schematically illustrated in FIG. 4.

The longitudinal fold lines 38 may be located at the same distance from each transverse side edge 5, 6, respectively. A transverse length 40 between each longitudinal fold line 38 and the respective transverse side edge 5, 6 may be 5-30% of the transverse length 33 between the side edges 5, 6 of the unfolded absorbent article. Moreover, the longitudinal fold lines 38 are preferably located transversally outside of the absorbent core 2 for avoiding unnecessary folding of the absorbent core 2.

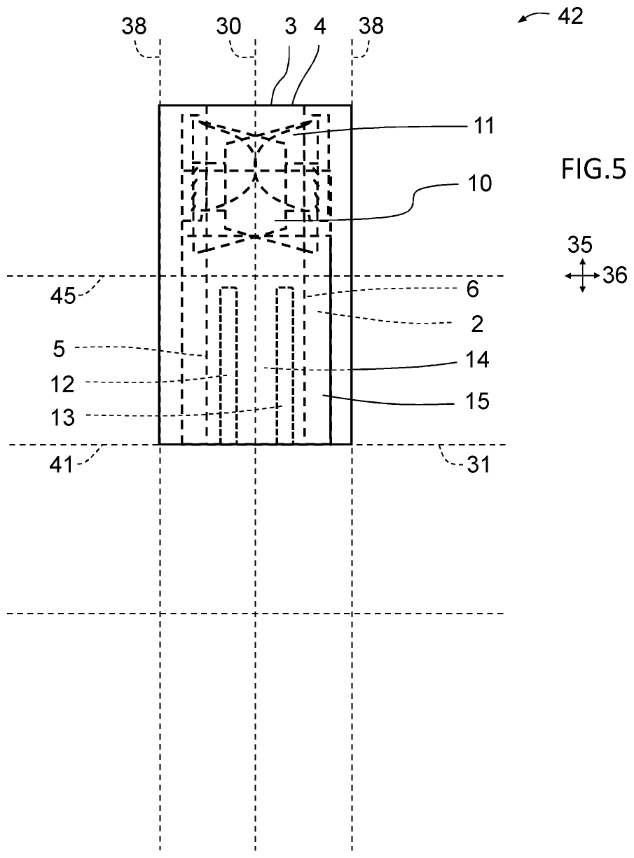

Thereafter, in a second folding step, the firstly folded absorbent article 39 is folded along a first transverse fold line 41 located in the region of the transverse center line 31 thereby forming a secondly folded absorbent article 42. The secondly folded absorbent article 42 is schematically illustrated in FIG. 5.

The secondly folded absorbent article 42 is considered to have two overlapping main sections of the absorbent article 1, namely a front section 43 and a back section 44. Having the first transverse fold line 41 located essentially aligned with the transverse center line 31 generally results in a minimal size of the secondly folded absorbent article 42 in the longitudinal direction. However, the first transverse fold line 41 may alternatively be located in the region of the transverse center line 31 that is slightly longitudinally offset from the transverse center line 31 for any reason. For example, the first transverse fold line 41 may displaced up to about 10% of the total length 32 of the absorbent article 1 from the transverse center line 31.

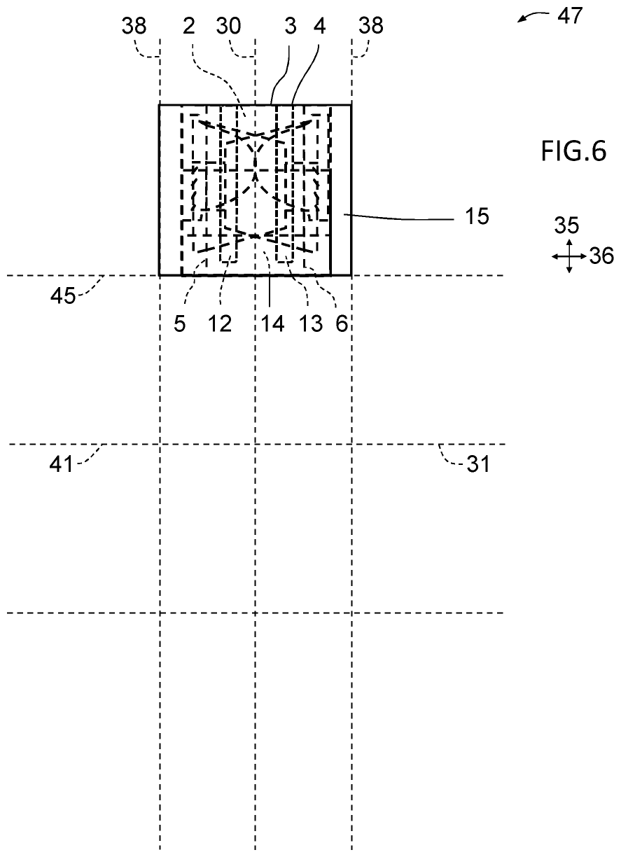

Thereafter, in a third folding step, the secondly folded absorbent article 42 is folded along a second transverse fold line 45 located approximately in the middle between the transverse center line 31 and a waist edge 3 thereby forming a thirdly folded absorbent article 47. The thirdly folded absorbent article 47 is schematically illustrated in FIG. 6.

The thirdly folded absorbent article 47 is considered to have four overlapping main sections of the absorbent article 1, namely twice the front section 43 and twice the back section 44. For this reason the thirdly folded absorbent article 47 is considered to have a so called quattro folding. The first folding step of folding each transverse side 37 of the absorbent article 1 along the respective longitudinal fold lines 38 is not reflected in the term quattro folding.

As shown in FIG. 3-6, the second transverse fold line 45 of the quattro folded absorbent article does not intersect a front portion of any of the first and second channels 12, 13. Instead, the second transverse fold line 45 is located in front of a front edge of the first and second channels 12, 13. Thereby, damages to the channels 12, 13 caused by the folding along the second transverse fold line 45 is avoided, and possible compressions of the absorbent core caused by the folding along the second transverse fold line 45 does not intersect and interfere with the desired functionality of channels 12, 13.

Similarly, as shown in FIG. 3-6, the second transverse fold line 45 of the quattro folded absorbent article does not intersect a back portion of any of the first and second channels 12, 13. Instead, the second transverse fold line 45 is located in front of a front edge of the first and second channels 12, 13. Thereby, damages to the channels 12, 13 caused by the folding along the second transverse fold line 45 is avoided, and possible compressions of the absorbent core caused by the folding along the second transverse fold line 45 does not intersect and interfere with the desired functionality of channels 12, 13.

The term “front portion” and “back portion” of the channels refers to the channels 12, 13 as such, and not to parts of the channels 12, 13 that are located in the front and back portions 7, 8 of the absorbent article 1.

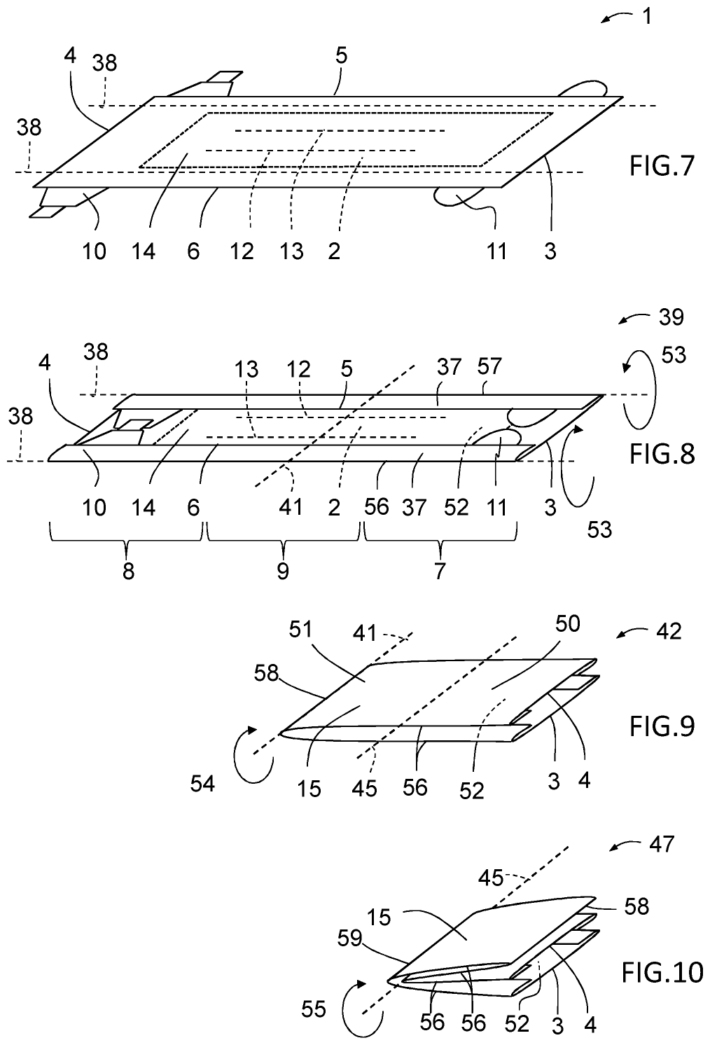

For better describing the folding sequence of FIG. 3-6 the substantially the same folding sequence is illustrated in a schematic 3D-view of the absorbent article in FIGS. 7-10. The absorbent article of FIGS. 3-6 has a similar structure, shape and composition as the absorbent article illustrated in FIGS. 7-10.

FIG. 7 shows the absorbent article 1 in an unfolded and planar state having the absorbent core 2 sandwiched between the topsheet 14 and backsheet 15, the front waist edge 3, back waist edge 4, and first and second side edges 5, 6. The absorbent core 2 comprises the first and second primarily longitudinally extending channels 12, 13.

In a first folding step the transverse sides 37 of the absorbent article 1 are folded along the longitudinal fold lines 38 to form the firstly folded absorbent article 39, as illustrated in FIG. 8. The folding operation is illustrated by first folding arrows 53 which show the folding of the transverse sides 37 along the longitudinal fold lines 38. The transverse side edges 56, 57 of the firstly folded absorbent article 39 are substantially coaxial with the longitudinal folds lines 38.

In the subsequent second folding step, the firstly folded absorbent article 39 is folded along the first transverse fold line 41 located in the region of the transverse center line to form the secondly folded absorbent article 42, which is illustrated in FIG. 9. The folding operation is illustrated by second folding arrow 54 that show the folding of the firstly folded absorbent article 39 along the first transverse fold line 41. The folded edge 58 in the crotch portion 9 is substantially coaxial with the first transverse fold line 41.

As clearly illustrated in FIG. 9, the topsheet 14 of the back portion 8 has been folded to face the topsheet 14 of the front portion 7 in the secondly folded absorbent article 42. Thereby, the backsheet 15 faces outwards of the secondly folded absorbent article 42 and the topsheet 14, which will face the skin of the user, is better protected from dirt etc., thereby providing a more hygienic absorbent article 1.

Moreover, this specific folding additionally provides a desirable pre-forming of the absorbent article 1, such that the absorbent article upon unpacking from the package has a curved natural shape with the topsheet facing a radially inner surface of such a curved natural shape.

Finally, in the third folding step, the secondly folded absorbent article 42 is folded along a second transverse fold line 45 located approximately in the middle between the transverse center line and the waist edge 3 thereby forming the thirdly folded absorbent article 47, which is illustrated in FIG. 10. The folding operation is illustrated by third folding arrow 55 that show the folding of the secondly folded absorbent article 42 along the second transverse fold line 45.

As discussed above, folding of the absorbent article 1 such that the inner surface of the back portion 8 faces the inner surface of the front portion 7 of the secondly folded absorbent article 42, and such that the outer surface 50 of the back portion 8 faces the outer surface 51 of the crotch portion 9 of the thirdly folded absorbent article 47, provides improved pre-forming of the absorbent article, wherein the pre-forming involves providing the unfolded absorbent article 1 with a step-wise curved shape for better fit on the curved natural shape of the body of the user between the stomach and back. The folds of the absorbent core 2 along both the first transverse fold line 41 and the forward of the second transverse fold line 45 results in a step-wise curved shape of the unfolded absorbent article with the topsheet 14 facing radially inwards in the curve, thereby providing improved fitting, comfort and leakage security of the article when carried by a user.

The circumferential edges of the quattro folded absorbent article of FIG. 10 are: on a first side the folded edge 59 located approximately in the middle between the transverse center line 31 and a waist edge 3 and substantially coaxial with the second transverse fold line 45, on a second side four layers of the transverse side edges 56 of the firstly folded absorbent article 39, on a third side the folded edge 58 in the crotch portion 9 that is located substantially overlapping with the front waist edge 3 and back waist edge 4, and on the fourth side the four layers of the transverse side edges 57 of the firstly folded absorbent article 39. Hence, all sides of the quattro folded absorbent article shown in FIG. 10 comprises folded portions of the absorbent article 1, thereby strengthening the absorbent article 1. The additional strengthening effect resulting from having four sections of the absorbent article in overlapping arrangement efficiently stabilizes weak areas within the absorbent article, such as area of the first and second channels 12, 13, and enables the folded absorbent article to be properly handled by the mechanical manufacturing and packaging devices with maintained proper position control of each individual absorbent article, such that high stacking quality is accomplished and packaging can be performed without having unacceptable amount of unintentional folds and wrinkles of the absorbent articles.

As clearly shown in FIGS. 9 and 10, folding of the secondly folded absorbent article 42 of FIG. 9 along the second transverse fold line 45 to form the thirdly folded absorbent article 47 involves folding the secondly folded absorbent article 42 such that an outer surface 50 of the back portion 8 comes to face an outer surface 51 of the crotch portion 9. This way of folding the secondly folded absorbent article 42 has several advantages. For example, the outer front region 52 of absorbent article 1 remains visible on the quattro folded absorbent article shown in FIG. 10. This is beneficial because the outer front region is typically used for informative printing, such as for example size of the absorbent article, and brand of the producer of the absorbent article.

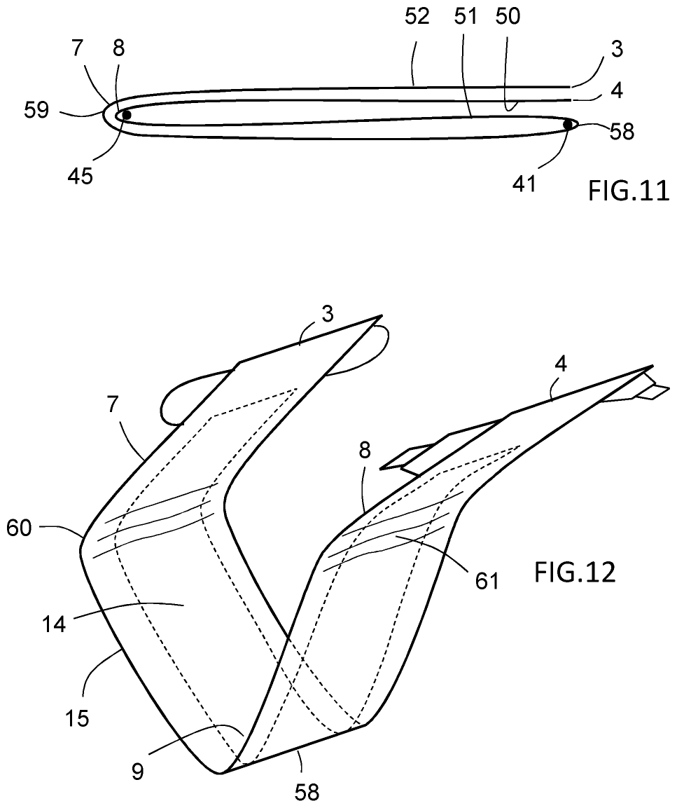

FIG. 11 illustrates schematically a side view of the quattro folded absorbent article of FIG. 10 having the outer front region 52 facing upwards in the figure, the first transverse fold line 41 located adjacent the folded edge 58 in the crotch portion 9 and the front waist edge 3 and back waist edge 4, the folded edge 59 located substantially coaxial with the second transverse fold line 45, and the outer surface 50 of the back portion 8 that is facing the outer surface 51 of the crotch portion 9. This folding is advantageous because the folding of the absorbent article 1 along second transverse fold line 45 results in a smaller radius fold of the back portion 8 and a larger radius fold of the front portion 7, as clearly illustrated in FIG. 11. Since the back portion 8 of the absorbent article typically has a smaller thickness than the front portion 7 the back portion can more easily be folded to have a smaller radius fold than the front portion without springing back to its unfolded state, and thus enabling forming an overall thinner folded absorbent article.

Moreover, a further advantage is improved pre-forming of the absorbent article 1 for better fit on the body of the user, as will be closed described below with reference to FIG. 12, which shows schematically a side view of the quattro folded absorbent article of FIG. 11 in a natural partly unfolded state.

A front fold 60 of the front portion 7 of the absorbent article 1 along the second transverse fold line 45 results in a desirable curve-forming effect of the absorbent article 1 because the front fold 60 assists a curved pre-forming with the topsheet 14 facing inwards, i.e. towards the radially inner side of the curve-shape. On the other hand, a back fold 61 of the back portion 8 of the absorbent article 1 along the second transverse fold line 45 results in an undesirable pre-forming effect of the back fold 61 because the resulting pre-forming is that the topsheet 14 faces outwards, i.e. towards the radially outer side of the curve at the back fold 61. However, the front fold 60 has a relatively strong desirable pre-forming effect on the article in the front 7, while the undesirable pre-forming effect of the back fold 61 is relatively weak due to lack, or at least smaller amount of absorbent core 2 in the back portion 8. Consequently, the quattro folding while having the outer surface 50 of the back portion 8 facing the outer surface 51 of the crotch portion 9 results in improved pre-forming of the folded absorbent article.

A further factor that generally has an effect on the pliability of the absorbent article 1 is thickness and density of the absorbent article 1 in the area of the absorbent core 2. A thinner absorbent article 1 typically results in reduced internal structural strength, thereby making the absorbent article 1 more floppy and pliable. Hence, a relative thin absorbent article 1 is an indication that the absorbent article 1 has a relatively high pliability.

Similarly, a relatively thin absorbent article 1 typically has a relatively high SAP/Pulp ratio, e.g. having generally a larger content of SAP than Pulp, in order to maintain the absorption capacity. However, SAP typically has a higher density than fluff pulp. Consequently, there is a relationship linking high density with thin and thus relatively pliable absorbent articles, wherein higher density in functionally linked with increased pliability, at least for absorbent articles with relatively high SAP/Pulp ratio.

According to the disclosure, the thickness of the absorbent article 1 in a region of the absorbent core 2 may be less than 7 mm, specifically less than 6 mm, and more specifically less than 5 mm. Theses increasingly narrow ranges indicate an increasingly pliable absorbent article 1.

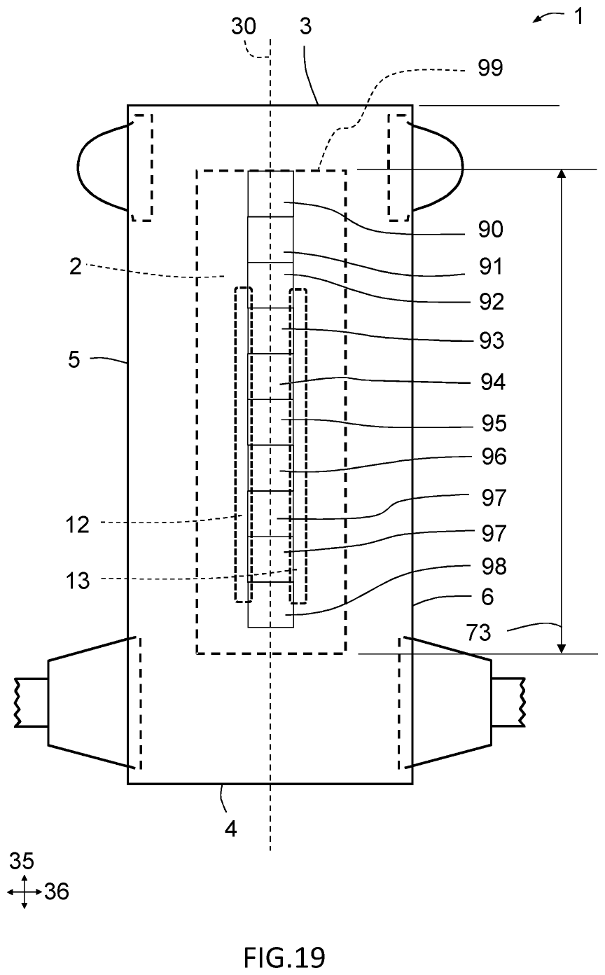

The thickness of the absorbent article 1 is measured in the area of the absorbent core 2 of the absorbent article 1. The method for measuring the thickness of the absorbent article is herein described with reference to FIG. 19, which shows the absorbent article in a flat state and with a row adjacent individual samples 90-98 of the absorbent article. The method for measuring the thickness of the absorbent article comprises: cutting off or neutralizing all elastic elements in the absorbent article 1, placing the absorbent article 1 in a flat and smooth state, punching out adjacent samples 90-99 of the absorbent article in the area of the absorbent core 2 starting from the front end 100 of the area of the absorbent core 2, which samples measure 3.75×3.75 cm and are located centered along the longitudinal center line 30.

Consequently, as an example, an article with a 40 cm long area of the absorbent core would thus yield 10 individual samples and with an extra 2.5 cm piece in the back that is discarded.

Moreover, in case a cut-out sample includes an area that is substantially void of absorbent material, or which has a thinner layer of absorbent material than adjacent areas of the absorbent core 2, such as a channel or a well, the sample must be discarded if the void or reduced thickness area amounts to 20% or more of the total sample area. The relevant parameters, i.e. thickness and density are then calculated from the remaining samples only.

The thickness is determined after 24 hours conditioning by placing the punched samples free and bare in a laboratory environment set to 23° C. and 50% relative humidity. The remaining part of the test method is performed in the same environment.

The thickness of the individual samples is measured under a 2.5 kPa pressure. The method for measuring the thickness includes: Lowering a circular foot with a 35 mm diameter slowly, and determine the thickness when the foot has rested on the sample for three seconds. The thickness of the absorbent article is subsequently determined by averaging the determined thickness of each sample, i.e. by calculate the average thickness for the samples.

According to the disclosure, the density of the absorbent article 1 in the region of the absorbent core is higher than 0.16 g/cm³, specifically higher than 0.18 g/cm³, and more specifically higher than 0.20 g/cm³.

The density of the absorbent article 1 is measured in the area of the absorbent core 2 of the absorbent article 1. First, an average sample weight is determined. This is performed by weighing all the 3.75×3.75 cm samples from the absorbent article (all together) on a balance (accurate to 0.001 g), and divide by the number of samples.

Finally, the average density (g/cm³) is calculated by dividing the average sample weight (g) by the product of the sample area (14.06 cm²) multiplied with the average sample thickness in (cm), i.e. the average density=average sample weight/(14.06×average sample thickness).

The thickness and density measurements are performed on five absorbent articles selected randomly from a consumer package. Alternatively, five absorbent articles are selected randomly from a line of 40 adjacent absorbent articles on a factory production line. The overall average, i.e. the average of the averages from the five individual absorbent articles, represents the thickness and density of any specific absorbent article model in the context of this disclosure.

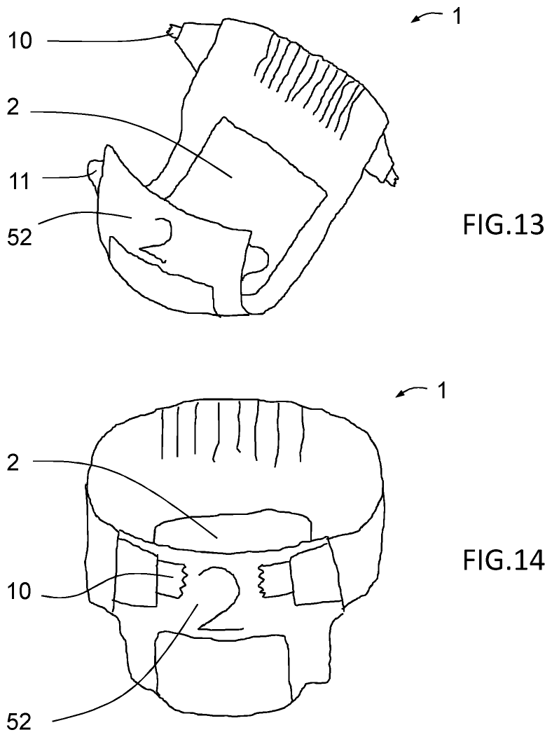

As schematically illustrated in FIGS. 13 and 14, the absorbent article may for example be an open baby diaper absorbent article 1, wherein FIG. 13 shows the article in an open state and FIG. 14 shows the article in a closed state, in which the fastening tabs are attached to a landing zone on the front portion 7.

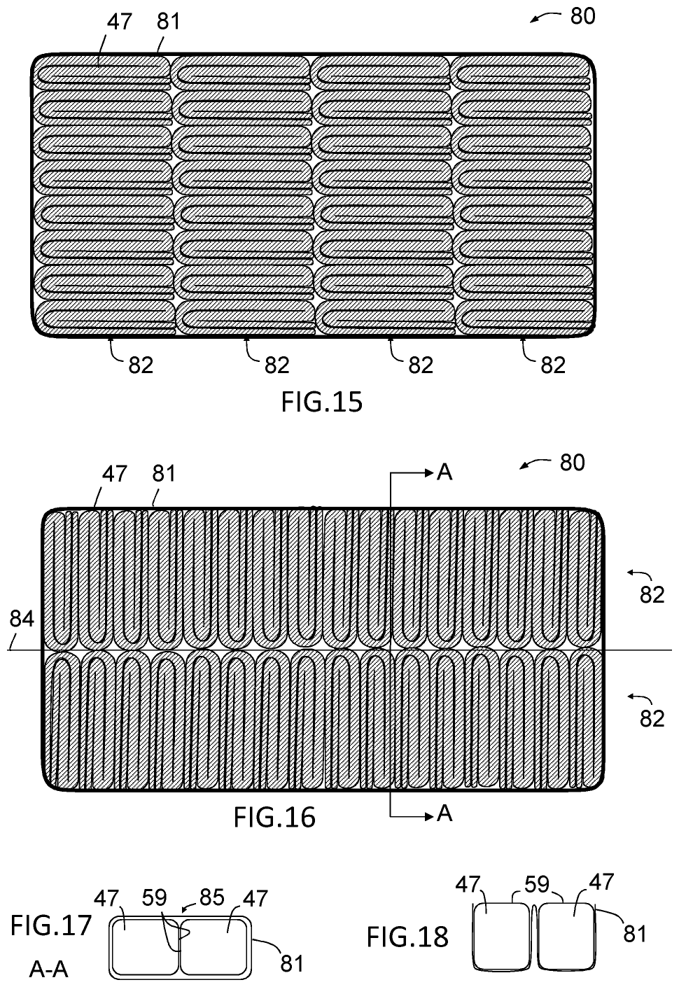

The disclosure further relates to a package 80 comprising a stack of folded absorbent articles 47 according to the disclosure above. FIGS. 15 and 16 illustrated schematically example packaging layouts of the folded absorbent articles 47 within a bag 81. FIG. 15 shows a package 80 that comprises four parallel stacks 82 of absorbent articles 1 located side-by-side. This packaging design enables by the relatively compact and small outer size of the quattro folded absorbent articles 47 increased stability and less amount of bag material per absorbent article, compared with a package with a single stack.

Alternatively, the quattro folded absorbent articles 47 may be packed in a package 80 that comprises two parallel stacks 82 of absorbent articles 1 located side-by-side. This type of packaging enables opening of the package 80 along a center line 84, for example by a weakening in the bag 81, and subsequently folding of the package along a fold line that is parallel with the center line 84.

For example, FIG. 17 schematically illustrates a cross-section along cut A-A in FIG. 16, wherein the quattro folded absorbent articles 47 of each stack 82 is arranged to have their folded edge 59 of the folded absorbent article 47 of FIG. 10 facing each other. A user may subsequently tear the bag 81 along a weakening 85 at center line 84 and thereafter fold the package such that two rows of folded absorbent articles 47 are visible and individual folded absorbent articles may be manually picked and removed from the package by grabbing the visible folded edge 59.

The disclosure further relates to a method for manufacturing a folded absorbent article 47, which absorbent article comprises, in an unfolded and planar state, a longitudinal center line, a transverse center line 30, a longitudinal length 32 extending from a front waist edge 3 to a back waist edge 4, and a transverse length 33 extending from a first side edge 5 to a second side edge 6, said absorbent article further comprising a front portion 7, a back portion 8 and a crotch portion 9, and fastening tabs 10 located on each transverse side of the back portion 8 for being releasably fastened to the front portion 7 when the absorbent article is in a fastened position The method comprises the steps of forming an absorbent core 2 comprising a first and a second primarily longitudinally extending channels 12, 13 located at least in the crotch portion 9; sandwiching the absorbent core 2 between a liquid-permeable topsheet 14 and a liquid-impermeable backsheet 15; folding each transverse side 37 of the absorbent article along respective longitudinal fold line 38, such that each of first and second transversely opposed side edges 5, 6 are folded onto a topsheet surface of the absorbent article thereby forming a firstly folded absorbent article 39; folding the firstly folded absorbent article 39 along a first transverse fold line 41 located in the region of the transverse center line 31 thereby forming a secondly folded absorbent article 42; and folding the secondly folded absorbent article 42 along a second transverse fold line 45 located approximately in the middle between the transverse center line 31 and a front waist edge 3 thereby forming a thirdly folded absorbent article 47.

Various types of materials may be used for the absorbent article 1. The topsheet 14 is arranged to face the wearer of the absorbent article 1 when worn. The topsheet 14 may be formed by a fluid permeable nonwoven fabric or film which is made of thermoplastic synthetic fibres. The topsheet 14 may be sufficiently liquid-permeable to allow discharged body fluids to penetrate through the thickness of the topsheet 14. Also, the topsheet 14 may be suitably manufactured from a material which is compliant and soft-feeling to the skin of the wearer. The topsheet 14 may consist of a single layer or have a laminate structure comprising a plurality of layers, for example, two or more layers. The layers may be made of the same material, or some or all the layers may be made of different materials.

The layer of the topsheet 14 or, for the case of a laminate structure, one, some, or all layers of the topsheet may be made of a single material or have plural portions made of different materials, e.g., within different parts of the wearer-facing surface of the topsheet.

The layer of the topsheet 14 or, for the case of a laminate structure, one, some or all layers of the topsheet may be a nonwoven material, a perforated plastic film, a plastic or textile mesh, or a liquid permeable foam layer.

The layer of the topsheet 14 or, for the case of a laminate structure, one, some, or all of the layers of the topsheet may be, for example, a hydrophilic, non-apertured nonwoven web of fibres, such as natural fibres, e.g., cotton or pulp fibres, synthetic fibres, e.g., polyester or polypropylene fibres, or a combination of these fibres.

The topsheet may have a basis weight in the range of 8-40 g/m². However, the disclosure is not limited to topsheets having this basis weight only.

Furthermore, the backsheet 15 may be constituted by a liquid-impermeable and breathable layer such as a polymeric film, for example a film of polyethylene or polypropylene. According to different embodiments, the materials which may be used for the backsheet 15 include thin and flexible fluid impermeable plastic films, or fluid impermeable nonwoven materials, fluid impermeable foams and fluid impermeable laminates.

The backsheet 15 may be formed by a single layer, but may alternatively be formed by a multi-layered structure, i.e. a laminate, wherein at least one layer is fluid impermeable. Furthermore, the backsheet 15 may be elastic in any direction.

Furthermore, the backsheet 15 may have a laminate structure comprising a liquid barrier sheet and a nonwoven layer arranged on top of each other (not shown in detail in the drawings), wherein the nonwoven layer is arranged at an outer side away from the wearer of the absorbent article 1 when worn.

The nonwoven layer may be made of thermoplastic polymer material fibres or filaments. The nonwoven layer may be formed by a variety of different processes, such as spunbonding, air laying, meltblowing or bonded carded web formation processes. The nonwoven layer may be made of an SMS (spunbond/meltblown/spunbond) or SS (spunbond/spunbond) nonwoven material of polypropylene or bicomponent fibres of polypropylene and polyethylene, or of a combination of such materials. The nonwoven layer may have a basis weight in the range of 5-40 g/m².

The liquid barrier sheet may be made of a plastic material, for example a thermoplastic film material, and/or a nonwoven material. For example, the liquid barrier sheet may be formed as a plastic layer, e.g., a thermoplastic layer, or a plastic film, e.g., a thermoplastic film. Forming the liquid barrier sheet of a plastic material, such as a thermoplastic film material, allows for a particularly good printability of the liquid barrier sheet.

The liquid barrier sheet may be a liquid impermeable, breathable or non-breathable layer. The liquid barrier sheet may consist of a single layer or have a laminate structure with a plurality of layers, e.g., two or more layers, three or more layers, or four or more layers. The layers of the liquid barrier sheet may be laminated, bonded or attached to each other, for example, by thermo and/or mechanical bonding, such as thermo-sealing, ultrasonic bonding, such as ultrasonic welding, an adhesive or adhesives, stitching or the like.

The liquid barrier sheet may be a breathable microporous film. The microporous film may be made of a material comprising at least two basic components, namely a thermoplastic elastomeric polyolefin polymer and a filler. These components and, in some embodiments, additional other components may be mixed together, heated and subsequently extruded into a mono-layer or multi-layer film using any one of various film-producing processes, such as cast embossed, chill and flat cast, and blown film processes.

Furthermore, the absorbent core 2 is provided between the topsheet 14 and the backsheet 15 to absorb the liquid, such as urine or other bodily fluids, which has passed through the topsheet 14. The absorbent core 2 may be made of one layer only, made from any suitable absorbent or liquid uptake material, such as one or more layers of cellulose fluff pulp, foam, fibre waddings or the like.

Each of the one or more absorbent layers of the absorbent core may have a homogeneous structure or a layered structure, i.e. an absorbent laminate of the same or different materials. Each of the one or more absorbent layers may have uniform or non-uniform thickness over the size of each respective absorbent layer. Similarly, the basis weight and composition may vary within the one or more absorbent layers. By way of example, an absorbent layer may comprise a mixture of absorbent and/or non-absorbent fibres and superabsorbent material, wherein the ratio of superabsorbent material to fibres may vary in the layer.

The absorbent core 2 may comprise suitable amounts of superabsorbent particles. Such superabsorbent material is well known in the field of absorbent articles, and is constituted by a water-swellable and water-insoluble material which is capable of absorbing large quantities of fluid upon formation of a hydrogel. The absorbent core 2 may contain superabsorbent material in the form of fibres or particles of absorbent polymer material. For example, the superabsorbent material may be surface cross-linked, partially neutralized polyacrylates.

The superabsorbent material, e.g., the superabsorbent fibres or particles, may be mixed with other absorbent or liquid uptake material or materials, such as cellulose fluff pulp, and/or arranged in pockets or layers in the absorbent core 2. The amount of superabsorbent material and pulp in the absorbent core 2 may 60-100% by weight superabsorbent material.

In other words, the absorbent core may, in a region of the absorbent core without a channel comprise about 60-100 wt % SAP (Super Absorbent Polymer). If the absorbent core in a region of the absorbent core without a channel comprises less than 100 wt % SAP the remaining material may for example be primarily pulp material, or the remaining material may for example be only pulp material.

The absorbent core 2 may further comprise components for improving the properties of the absorbent core 2. For example, the absorbent core 2 may comprise a binder or binders, such as binder fibres.

Furthermore, as known by the skilled person, the various layers of the absorbent article 1 may be attached by adhesive material. Such adhesive is not shown in the drawings.

One or more additional layers may be provided in the absorbent article 1. For example, an acquisition layer may be arranged between the absorbent core 2 and the topsheet 14. Such an additional layer may for example be in the form of an airlaid layer, a spunlaced layer, a high-loft, foam or any other type of material layer which may be used in an absorbent article to act as a liquid acquisition and absorption layer. The acquisition layer is adapted to quickly receive and temporarily store discharged liquid before it is absorbed by the absorbent core. Such acquisition layer may be composed of for example airlaid nonwoven, spunlaced nonwoven, high loft nonwoven or foam materials. An airlaid nonwoven may be produced with fluff, wood pulp, and here the fluff fibres are dispersed into a fast-moving air stream and condensed onto a moving screen by pressure and vacuum.

The absorbent core 2 may be wrapped in nonwoven material and positioned between the topsheet 14 and the backsheet 15 during manufacturing of the absorbent article 1.

Furthermore, the absorbent core 2 and/or the topsheet 14 may comprise at least one additive material such as a skin care composition.

Although the disclosure has been described in relation to specific combinations of components, it should be readily appreciated that the components may be combined in other configurations as well which is clear for the skilled person when studying the present application. Thus, the above description of the example embodiments and the accompanying drawings are to be regarded as a non-limiting example of the disclosure and the scope of protection is defined by the appended claims. The disclosure may be varied from the examples provided while remaining within the scope of the claims. For example, the materials and dimensions used for the different layers forming the absorbent article 1 may be varied, as indicated above. The absorbent article may further include leg elastics, standing gathers, crotch and waist elastics, side panels, fastening systems etc. as known to the skilled man in the art and depending of the type of absorbent article intended.

What is claimed is:

1. A folded absorbent article comprising: an absorbent core sandwiched between a liquid-permeable topsheet and a liquid-impermeable backsheet, the absorbent article comprising, in an unfolded and planar state, a longitudinal center centre line a transverse center centre line, a longitudinal length extending from a front waist edge to a back waist edge, and a transverse length extending from a first side edge to a second side edge, said absorbent article further comprising a front portion, a back portion and a crotch portion, and fastening tabs located on each transverse side of the back portion for being releasably fastened to the front portion when the absorbent article is in a fastened position,
wherein said absorbent core comprises a first primarily longitudinally extending channel and a second primarily longitudinally extending channel located at least in the crotch portion,
wherein each transverse side of the absorbent article is folded along respective longitudinal fold line, such that each of first and second transversely opposed side edges are folded onto a topsheet surface of the absorbent article thereby forming a firstly folded absorbent article,
wherein the firstly folded absorbent article is folded along a first transverse fold line located in a region of the transverse center line thereby forming a secondly folded absorbent article, and
wherein the secondly folded absorbent article is folded along a second transverse fold line located approximately in the middle between the transverse center line and a waist edge thereby forming a thirdly folded absorbent article.

2. The folded absorbent article according to claim 1, wherein the second transverse fold line does not intersect a front portion of any of the first and second channels.

3. The folded absorbent article according to claim 1, wherein the second transverse fold line does not intersect a back portion of any of the first and second channels.

4. The folded absorbent article according to claim 1, wherein an inner surface of the back portion faces an inner surface of the front portion of the secondly folded absorbent article, and wherein an outer surface of the back portion faces an outer surface of the crotch portion of the thirdly folded absorbent article.

5. The folded absorbent article according to claim 1, wherein at least one of the first and second channels has a longitudinal length which is between 5-50%, of a total longitudinal length of the absorbent article, and
wherein at least one of the first and second channels has a longitudinal length which is between 10-60% of a longitudinal length of the absorbent core.

6. (canceled)

7. The folded absorbent article according to claim 1, wherein a longitudinal distance between the front waist edge of the absorbent article and a front edge of at least one of the first and second channels is between 15-40%, of a total longitudinal length of the absorbent article.

8. The folded absorbent article according to claim 1, wherein the first and second channels are generally straight.

9. The folded absorbent article according to claim 1, wherein the first and second channels are generally parallel to said longitudinal center line.

10. The folded absorbent article according to claim 1, wherein the first and second channels constitute sections of the absorbent core which:
are generally free from absorbent material, or
have a thinner layer of absorbent material than adjacent areas of the absorbent core.

11. The folded absorbent article according to claim 1, wherein each of the first and second channels has a width of at least 3 mm.

12. The folded absorbent article according to claim 1, wherein the absorbent core comprises further primarily longitudinally extending channels located at least in the crotch portion.

13. The folded absorbent article according to claim 1, wherein the absorbent core in a region of the absorbent core without a channel comprises 60-100 wt % super absorbent polymer (SAP).

14. The folded absorbent article according to claim 1, wherein a thickness of the absorbent article in a region of the absorbent core is less than 7 mm.

15. The folded absorbent article according to claim 1, wherein a density of the absorbent article in a region of the absorbent core is higher than 0.16 g/cm³.

16. The folded absorbent article according to claim 1, wherein the absorbent core is constituted of one single core layer.

17. The folded absorbent article according to claim 1, wherein the absorbent core has a generally rectangular shape shaped.

18. The folded absorbent article according to claim 1, wherein the absorbent article is an open baby diaper absorbent article.

19. A package comprising:
at least two parallel stacks of folded absorbent articles according to claim 1.

20. (canceled)

21. A method for manufacturing a folded absorbent article, the absorbent article comprising, in an unfolded and planar state, a longitudinal center line, a transverse center line, a longitudinal length extending from a front waist edge to a back waist edge, and a transverse length extending from a first side edge to a second side edge, the absorbent article further comprising a front portion, a back portion and a crotch portion, and fastening tabs located on each transverse side of the back portion for being releasably fastened to the front portion when the absorbent article is in a fastened position, the method comprising:
forming an absorbent core comprising a first primarily longitudinally extending channel and a second primarily longitudinally extending channel located at least in the crotch portion;
sandwiching the absorbent core between a liquid-permeable topsheet and a liquid-impermeable backsheet,
folding each transverse side of the absorbent article along respective longitudinal fold line, such that each of first and second transversely opposed side edges are folded onto a topsheet surface of the absorbent article thereby forming a firstly folded absorbent article
folding the firstly folded absorbent article along a first transverse fold line located in a region of the transverse center line thereby forming a secondly folded absorbent article and
folding the secondly folded absorbent article along a second transverse fold line located approximately in the middle between the transverse center line and a waist edge thereby forming a thirdly folded absorbent article.

22. The folded absorbent article according to claim 2, wherein the second transverse fold line does not intersect a back portion of any of the first and second channels,
wherein an inner surface of the back portion faces an inner surface of the front portion of the secondly folded absorbent article, and wherein an outer surface of the back portion faces an outer surface of the crotch portion of the thirdly folded absorbent article,
wherein at least one of the first and second channels has a longitudinal length which is between 28-38% of a total longitudinal length of the absorbent article,
wherein at least one of the first and second channels has a longitudinal length which is between 30-50% of a longitudinal length of the absorbent core,
wherein a longitudinal distance between the front waist edge of the absorbent article and a front edge of at least one of the first and second channels is between 22-25% of a total longitudinal length of the absorbent article,
wherein the first and second channels are generally straight,
wherein the first and second channels are generally parallel to said longitudinal center line,
wherein the first and second channels constitute sections of the absorbent core which:
are generally free from absorbent material, or
have a thinner layer of absorbent material than adjacent areas of the absorbent core,
wherein each of the first and second channels has a width of at least 5 mm,
wherein the absorbent core comprises further primarily longitudinally extending channels located at least in the crotch portion,
wherein the absorbent core in a region of the absorbent core without a channel comprises 60-100 wt % super absorbent polymer (SAP),
wherein a thickness of the absorbent article in a region of the absorbent core is less than 5 mm,
wherein a density of the absorbent article in a region of the absorbent core is higher than 0.20 g/cm³,
wherein the absorbent core is constituted of one single core layer,
wherein the absorbent core has a generally rectangular shape, and
wherein the absorbent article is an open baby diaper absorbent article.
